(19) United States
(12) Patent Application Publication
(10) Pub. No.: US 2003/0147832 A1
(43) Pub. Date: Aug. 7, 2003

(54) Polymer comprising water-soluble unit and unit with an icst, and aqueous composition comprising it

(76) Inventor: Florence L'Alloret, Boussingault (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/070,910

(22) PCT Filed: Jan. 14, 2002
(86) PCT No.: PCT/FR02/00124

(51) Int. Cl. A61K 7/06; A61K 7/11; C08J 3/28

(52) U.S. Cl. 424/70.15; 522/152

(57) ABSTRACT

Polymer comprising water-soluble units and units with an LCST, and aqueous composition comprising it.

The present application relates to a polymer comprising water-soluble units and units having a temperature of LCST type consisting of N-vinylcaprolactam homopolymers or of derived copolymers.

The invention also relates to an aqueous composition, especially a thickened, or even gelled, transparent aqueous composition, comprising at least one such polymer and an aqueous phase, for example a cosmetic or dermatological composition.

### DESCRIPTION

The present invention relates to a novel family of polymers that may be used in cosmetic or dermatological compositions, especially to modify their Theological properties.

The thickeners usually used in cosmetics or pharmaceuticals to control the rheology of compositions, especially aqueous compositions, generally undergo a reduction in viscosity when the temperature of the medium increases.

However, this behaviour can present certain drawbacks, such as the change in the rheology of the composition as a function of changes in temperature (compositions which become fluid in summer and are more viscous in winter).

Particular polymers whose solubility in water is modified above a certain temperature are known in the prior art. These are polymers with a heat-induced demixing temperature (or cloud point) thus defining their region of water solubility. The minimum demixing temperature obtained as a function of the polymer concentration is known as the “LCST” (Lower Critical Solution Temperature).

Some of these polymers are especially described in the articles by Taylor et al, Journal of Polymer Science, part A; Polymer Chemistry, 1975, 13, 2551; by Bailey et al, Journal of Applied Polymer Science, 1959, 1,56; and by Heskins et al, Journal of Macromolecular Science, Chemistry A2, 1968, 8, 1441.

In addition, polymers comprising, based on those of the invention, water-soluble units and units with an LCST and having heat-induced gelling properties observed above the demixing temperature of the chains with an LCST are described in the following documents.

The document by D. Hourdet et al., Polymer, 1994, Vol. 35, No. 12, pages 2624-2630 [1] relates to the reversible heat-induced thickening of aqueous solutions of copolymer comprising a water-soluble polyacrylic acid backbone with poly(ethylene oxide) (PEO) grafts.

The document by F. L'Alloret et al., Coll. Polym. Sci., 1995, Vol. 273, No. 12, pages 1163-1173 [2] relates to the heat-induced thickening behaviour in aqueous solution of polymers comprising a 2-acrylamido-2-methylpropanesulphonic acid (AMPS) backbone and poly(ethylene oxide) side chains.

Similarly, the document by F. L'Alloret, Revue de l'Institut Francais du Pétrole [Review of the French Petroleum Institute], 1997, Vol. 52, No. 2, pages 117-128 [3] describes the reversible heat-induced association of copolymers with a water-soluble polyacrylic backbone or based on AMPS with PEO grafts.

Polymers such as those mentioned in documents [1], [2] and [3] find their use, in particular, in the petroleum industry.

Thus, document [4] describes thermoviscosifying polymers with a water-soluble backbone comprising segments with an LCST, or bearing side chains with an LCST, which may be used especially as thickeners, constituents of drilling fluids or other fluids, and industrial cleaning fluids.

Document [5] describes polymers similar to those of document [4] and their use as anti-sedimentation agents for suspensions, optionally in cosmetic preparations.

Moreover, it is known, especially from patent application WO-95/24430, to use such heat-sensitive and pH-sensitive polymers in cosmetics or pharmaceuticals. The polymers described in the said patent application may be of any chemical nature; in particular, they may be in the form of grafted copolymers comprising a pH-sensitive backbone with heat-sensitive grafts; or, conversely, in the form of a heat-sensitive backbone bearing pH-sensitive grafts; or alternatively in the form of block copolymers formed from pH-sensitive units and heat-sensitive units.

These heat-sensitive blocks or grafts may be prepared by polymerization of vinyl monomers or by polymerization of cyclic ether monomers. In particular, these grafts or blocks may be in the form of poly(N-alkyl substituted)acrylamides or of block copolymers of ethylene oxide and of propylene oxide.

However, the heat-induced gelling properties of these polymers lead to opaque gels, as is pointed out in the description of the said patent application. Now, this opacification of the aqueous solutions may be an unacceptable drawback for uses in cosmetics.

In point of fact, the polymer in the said document is fundamentally different from that of the invention since it has overall for the entire polymer an LCST in the temperature range from 20 to 40° C., unlike the polymer of the invention, which comprises units with an LCST, but does not itself have an LCST. The polymer of the invention does not lose its transparency on heating and is soluble in water irrespective of the temperature (between 5 and 80° C.); the expression “soluble in water” means that it has a solubility of at least 10 g/l and preferably of at least 20 g/l.

This is likewise the case with respect to patent application WO-97/00275, which describes in many cosmetic applications the use of polymers with heat-sensitive units, that are in the form of block copolymers of ethylene oxide and of propylene oxide. Moreover, in the said patent application, it is not easy to control the structure or chemical nature of the synthesized polymers.

The aim of the present invention is to overcome the drawbacks of the prior art and to propose a novel family of polymers for controlling the rheology of aqueous compositions as a function of the temperature, while at the same time maintaining a certain level of transparency for the compositions.

Moreover, the chemical structure of this novel family of polymers makes it possible firstly to prepare them on demand, irrespective of the nature of the polymer backbone and/or of the grafts, and/or their respective amount and/or the desired structure (for example linear or branched); this especially makes it possible to adapt the desired properties, as a function of the intended applications. Furthermore, these novel polymers are biocompatible, unlike poly(N-alkyl-substituted acrylamide) derivatives.

A first object of the present invention is a polymer comprising water-soluble units and units having a temperature of LCST type, said polymer being obtainable by reaction between the reactive sites, firstly of the water-soluble units bearing, before reaction, at least two reactive sites, and secondly of the units with an LCST bearing, before reaction, at least one reactive site, so as to form a covalent bond, said units with an LCST consisting of N-vinylcaprolactam homopolymers or of copolymers derived therefrom, the proportion by mass of the units with an LCST in the polymer being between 5% and 70%.

Another object of the invention is a thickened, or even gelled, transparent aqueous composition comprising at least one polymer as defined above.

Water-soluble polymers are thus obtained, especially having a solubility in water, at 20° C., of at least 10 g/l and preferably of at least 20 g/l. These polymers do not have an LCST from 5 to 80° C.

These water-soluble polymers make it possible to control the rheology of aqueous compositions as a function of the temperature, while at the same time maintaining the transparency of the said compositions.

Moreover, it is possible to adjust as desired the relevant temperature range to the intended cosmetic application, by appropriately selecting the chemical nature of the water-soluble units, of the units with an LCST, and also the respective amounts thereof.

The polymers according to the present invention may be block polymers or grafted polymers, which comprise, on the one hand, water-soluble units, and on the other hand, units having a temperature of LCST type as defined below.

The polymers used in the context of the invention may thus be block polymers comprising, for example, water-soluble blocks alternating with blocks with an LCST.

These polymers may also be in the form of grafted polymers whose backbone is formed from water-soluble units, bearing grafts with an LCST. This structure may be partially crosslinked.

The expression “water-soluble units” means units that are soluble in water, at 20° C., to a proportion of at least 10 g/l and preferably of at least 20 g/l.

However, water-soluble units that may also be used include units not necessarily having the solubility mentioned above, but which, in solution at 1% by weight in water at 20° C., allow the production of a solution that is macroscopically homogeneous and transparent, that is to say having a maximum light transmittance value, irrespective of the wavelength of between 400 and 800 nm, through a sample 1 cm thick, of at least 80% and preferably of at least 85%.

The water-soluble units may be in the form of blocks within a block polymer, or may constitute the backbone of a grafted polymer.

These water-soluble units do not have a heat-induced demixing temperature of LCST type.

The water-soluble units may be obtained by free-radical polymerization of vinyl monomers, or by poly-condensation, or alternatively may consist of existing natural polymers or modified natural polymers.

In all cases, the water-soluble units used bear, before reaction, at least two reactive sites.

Examples which may be mentioned include the monomers A below, which may be used to form water-soluble units, alone or as a mixture:

(meth)acrylic acid,

vinyl monomers of formula (Ia) below:

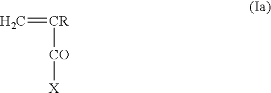

in which:

R is chosen from H, —CH₃, —C₂H₅ or —C₃H₇

X is chosen from:

alkyl oxides of —OR′ type in which R′ is a linear or branched, saturated or unsaturated hydrocarbon-based radical containing from 1 to 6 carbons, substituted with at least one hydroxyl (—OH); primary amine (—NH₂); secondary amine (—NHR₁) or tertiary amine (—NR₁R₂) group, with R₁ and R₂, independently of each other, representing a linear or branched, saturated or unsaturated hydrocarbon-based radical containing 1 to 25 carbon atoms, with the proviso that the sum of the carbon atoms of R₁+R₂ does not exceed 26; a halogen atom (iodine, bromine, chlorine or fluorine);

groups —NH₂, —NHR′ and —NR′R″ in which R′ and R″ are, independently of each other, linear or branched, saturated or unsaturated hydrocarbon-based radicals containing 1 to 25 carbon atoms, with the proviso that the total number of carbon atoms of R′+R″ does not exceed 26, the said R′ and R″ optionally being substituted with a hydroxyl (—OH); sulphonic (—SO₃⁻); sulphate (—SO₄⁻); phosphate (—PO₄H₂); primary amine (—NH₂); secondary amine (—NHR₁), tertiary amine (—NR₁R₂) and/or quaternary amine (—N⁺R₁R₂R₃) group, with R₁, R₂ and R₃ being, independently of each other, a linear or branched, saturated or unsaturated hydrocarbon-based radical containing 1 to 25 carbon atoms, with the proviso that the sum of the carbon atoms of R₁+R₂ does not exceed 26, and that the sum of the carbon atoms of R₁+R₂+R₃ does not exceed 27;

maleic anhydride;

itaconic acid;

vinyl alcohol of formula CH₂═CHOH;

vinyl acetate of formula CH₂═CH—OCOCH₃.

In addition to the monomers A mentioned above, which allow, alone or as a mixture, the production of a water-soluble unit having, before reaction, at least two reactive sites, it is possible to use, in combination with these monomers A, other monomers B that do not, by themselves, allow the production of a water-soluble unit having, before reaction, a reactive site.

Among the monomers B that may be mentioned, alone or as a mixture, are:

vinyl monomers of formula (Ib) below:

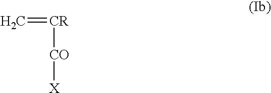

in which:

R is chosen from H, —CH₃, —C₂H₅ or —C₃H₇;

X is chosen from alkyl oxides of —OR′ type in which R′ is a linear or branched, saturated or unsaturated hydrocarbon-based radical containing from 1 to 6 carbons, optionally substituted with a halogen atom (iodine, bromine, chlorine or fluorine), a sulphonic (—SO₃⁻), sulphate (—SO₄⁻), phosphate (—PO₄H₂); and/or quaternary amine (—N⁺R₁R₂R₃) group, with R₁, R₂ and R₃ being, independently of each other, a linear or branched, saturated or unsaturated hydrocarbon-based radical containing 1 to 25 carbon atoms, with the proviso that the sum of the carbon atoms of R₁+R₂+R₃ does not exceed 27;

N-vinyllactams such as N-vinylpyrrolidone, N-vinylcaprolactam and N-butyrolactam;

vinyl ethers of formula CH₂═CHOR in which R is a linear or branched, saturated or unsaturated hydrocarbon-based radical containing from 1 to 25 carbons;

styrene and its derivatives, especially styrene sulphonate;

dimethyldiallylammonium chloride;

vinylacetamide.

Among the polycondensates and the natural polymers or modified natural polymers that may constitute all or part of the water-soluble units, mention may be made of:

water-soluble polyurethanes having, before reaction, at least two reactive sites, especially bearing carboxylic acid functions;

xanthan gum, especially the product sold under the names Keltrol T and Keltrol SF by Kelco; or Rhodigel SM and Rhodigel 200 from Rhodia;

alginates (Kelcosol from Monsanto) and derivatives thereof such as propylene glycol alginate (Kelcoloid LVF from Kelco);

cellulose derivatives and especially carboxymethylcellulose, hydroxypropylcellulose, hydroxyethylcellulose and quaternized hydroxyethylcellulose;

galactomanans and derivatives thereof, such as Konjac gum, guar gum, hydroxypropylguar, hydroxypropylguar modified with sodium methylcarboxylate groups hydroxypropyl-trimethylammonium guar chloride.

Mention may also be made of polyethyleneimine.

The water-soluble units preferably have a molar mass of between 5000 g/mol and 5 000,000 g/mol when they constitute the water-soluble backbone of a grafted polymer.

These water-soluble units preferably have a molar mass of between 5 000 g/mol and 100 000 g/mol when they constitute a block of a multiblock polymer.

As has been defined above, the water-soluble units bear, before reaction, at least two reactive sites, capable of reacting with at least one reactive site borne, before reaction, by the units with an LCST, so as to give a covalent bond in the final polymer of the invention.

This reactive site, before reaction, may be chosen especially from alcohol, isocyanate, primary, secondary or tertiary amine, carboxylic acid and halogen functions.

In particular, a reactive site of the carboxylic acid type will generally react with a reactive site of the alcohol or amine type; an isocyanate site will rather react with an alcohol site, and a halogen site will rather react with an alcohol or amine site.

In the final polymer according to the invention, there will thus be water-soluble units derived from water-soluble units bearing, before reaction, reactive sites, and units with an LCST which are derived from the units with an LCST bearing, before reaction, reactive sites.

The units with an LCST are linked to the water-soluble units via linking groups derived from the reaction of the reactive sites borne, before reaction, on the one hand by the water-soluble units, and on the other hand by the units with an LCST. These linking groups will be, for example, ester, amide or ether groups.

It should be noted that the definition both of the water-soluble units and of the units with an LCST forming part of the composition of the final polymer according to the invention does not, in principle, comprise these linking groups.

The expression “units with an LCST” means units whose water solubility is modified beyond a certain temperature. They are units with a heat-induced demixing temperature (or cloud point) defining their region of solubility in water. The minimum demixing temperature obtained as a function of the polymer concentration is referred to as the “LCST” (Lower Critical Solution Temperature). For each polymer concentration, this heat-induced demixing temperature is observed; it is higher than the LCST, which is the minimum point of the curve. Below this temperature, the polymer is soluble in water; above this temperature, the polymer loses its solubility in water.

The expression “soluble in water” means that the units have a solubility at 20° C. of at least 1 g/l and preferably of at least 2 g/l.

The measurement of the LCST may be performed visually: the temperature at which the cloud point of the aqueous solution appears is determined; this cloud point is reflected by the opacification of the solution, or the loss of transparency.

In general, a transparent composition will have a maximum light transmittance value, irrespective of the wavelength of between 400 and 800 mm, through a sample 1 cm thick, of at least 80% and preferably of at least 90% (see EP-A-0 291 334).

The transmittance may be measured by placing a sample 1 cm thick in the light beam of a spectrophotometer working at the wavelengths of the light spectrum.

The units with an LCST used in the present invention consist of N-vinylcaprolactam homopolymers of formula (II) below:

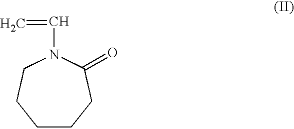

or of copolymers of N-vinylcaprolactam and:

of a vinyl monomer of formula (III) below:

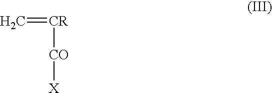

in which:

R is chosen from H, —CH₃, —C₂H₅ or —C₃H₇; and

X is chosen from:

alkyl oxides of —OR′ type in which R′ is a linear or branched, saturated or unsaturated hydrocarbon-based radical containing from 1 to 6 carbons, optionally substituted with at least one halogen atom (iodine, bromine, chlorine or fluorine); a sulphonic (—SO₃⁻), sulphate (—SO₄⁻), phosphate (—PO₄H₂); hydroxyl (—OH); primary amine (—NH₂); secondary amine (—NHR₁), tertiary amine (—NR₁R₂) or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R₁+R₂+R₃ does not exceed 6; and

groups —NH₂, —NHR′ and —NR′R″ in which R′ and R″ are, independently of each other, linear or branched, saturated or unsaturated hydrocarbon-based radicals containing 1 to 6 carbon atoms, with the proviso that the total number of carbon atoms of R′+R″ does not exceed 7, the said R′ and R″ optionally being substituted with a halogen atom (iodine, bromine, chlorine or fluorine); a hydroxyl (—OH); sulphonic (—SO₃⁻), sulphate (—SO₄⁻); phosphate (—PO₄H₂); primary amine (—NH₂); secondary amine (—NHR₁), tertiary amine (—NR₁R₂) and/or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R″+R₁+R₂+R₃ does not exceed 7;

maleic anhydride;

itaconic acid;

vinyl alcohol of formula CH₂═CHOH; vinyl acetate of formula CH₂═CH—OCOCH₃;

a vinyl ether of formula CH₂═CHOR in which R is a linear or branched, saturated or unsaturated hydrocarbon-based radical containing from 1 to 6 carbons;

a water-soluble styrene derivative, especially styrene sulphonate;

dimethyldiallylammonium chloride; and

vinylacetamide.

Preferably, the molar mass of the units with an LCST is between 1 000 and 500 000 g/mol and especially between 2 000 and 50 000 g/mol.

Needless to say, these units with an LCST also need to bear, before reaction, at least one reactive site capable of reacting with the reactive site borne, before reaction, by the water-soluble units, so as to form a covalent bond.

As previously, this reactive site, before reaction, may be chosen from alcohol, isocyanate, primary, secondary or tertiary amine, carboxylic acid and halogen functions.

When the final polymer consists of a water-soluble backbone and grafts with an LCST, the reactive sites, before reaction, are randomly distributed along the water-soluble backbone formed by the water-soluble units and those, before reaction, of the grafts are located on at least one of the ends of the chains with an LCST of the units with an LCST, before reaction.

When the polymer is of multiblock type, the reactive sites, before reaction, are located at the ends of the water-soluble units and of the units with an LCST.

The units with an LCST, especially before reaction, may thus especially be in the form of N-vinylcaprolactam homopolymers or of amino, especially monoamino, diamino or triamino, derivative copolymers.

The synthesis of these compounds may be performed by free-radical polymerization using a pair of initiators such as aminoethanethiol hydrochloride in the presence of azobis(isobutyronitrile) so as to obtain oligomers with an LCST having, before reaction, an amino reactive end.

Among the final polymers that may be used in the context of the invention, mention may be made in particular of:

polymers whose backbone consists of:

a linear acrylic acid homopolymer;

a linear copolymer of acrylic acid and of AMPS, and/or of acrylamide;

a crosslinked homopolymer of polyacrylic acid, and/or of AMPS;

a natural derivative such as xanthan gum, alginates, carboxymethylcellulose or hydroxy-propylguar modified with sodium methyl-carboxylate groups;

and bearing grafts with an LCST consisting of N-vinylcaprolactam homopolymers or derived copolymers, these copolymers bearing, before reaction, reactive sites, and being, for example, amino, especially monoamino, diamino or triamino.

The proportion by mass of the units with an LCST in the final polymer is between 5% and 70%, preferably between 20% and 65% and especially between 30% and 60% by weight relative to the final polymer.

Preferably, the heat-induced demixing temperature of LCST type of the said units with an LCST is between 5° C. and 40° C. and preferably between 10° C. and 35° C., for a concentration by mass in water of 1% by weight of the said units with an LCST.

The polymers used in the context of the invention may be readily prepared by a person skilled in the art on the basis of his general knowledge.

In particular, when the final polymer is in the form of a grafted polymer especially containing a water-soluble backbone with grafted units with an LCST, it is possible to prepare it by grafting the chains with an LCST having, before reaction, at least one reactive end, especially an amino end, onto the water-soluble polymer forming the backbone, the said polymer bearing at least 10% (on a molar basis) of carboxylic acid groups.

This reaction may be carried out in the presence of a carbodiimide such as dicyclohexylcarbodiimide or 1-(3-dimethylaminopropyl)-3-ethylcarbodiimide hydrochloride, in a solvent such as N-methylpyrrolidone or water.

To do this, inspiration may be taken from the preparation method described in the publication by Hourdet et al., Polymer, Vol. 38, No. 10, pp. 2535-2547, 1997.

Another possibility for preparing grafted polymers consists in copolymerizing, for example, a macromonomer with an LCST (chain with an LCST described above with a vinyl end) and a water-soluble vinyl monomer of formula (Ia) or (Ib).

When the final polymer is in the form of a block polymer, it is possible to prepare it by coupling between water-soluble units and units with an LCST having, before reaction, complementary reactive sites at each end.

The polymers thus obtained are water-soluble heat-induced gelling and biocompatible.

They allow the production of thickened, or even gelled, aqueous compositions that especially have a viscosity which is constant or which increases when the temperature increases, and which moreover have good transparency.

In the context of the present invention, the expression “transparent solution or composition” has the standard definition given in the dictionary. Thus, a transparent composition readily allows light to pass through it and allows objects to be clearly distinguished through its thickness.

The transmittance may be measured by placing a sample 1 cm thick in the light beam of a spectrophotometer working in the wavelengths of the light spectrum.

In particular, the compositions thus prepared may have a maximum light transmittance value, irrespective of the wavelength of between 400 and 800 nm, through a sample 1 cm thick, of at least 80% and preferably of at least 85% (see EP-A-0 291 334).

The polymers according to the invention are preferably present in the aqueous compositions in an amount preferably of between 0.01% and 20% by weight, especially from 0.05% to 15% by weight and in particular from 0.1% to 10% by weight.

These compositions and the polymers they comprise find a most particular application in cosmetics and dermatology.

The said composition comprises, in addition to the polymer as defined above, an aqueous phase, which may comprise, in addition to water, a floral water such as cornflower water, a mineral water such as eau de Vittel, eau de Lucas or eau de La Roche Posay and/or a spring water.

It is possible to add to the said aqueous composition the constituents usually used in the intended type of application. Needless to say, a person skilled in the art will take care to select these optional additional constituents, and/or the amount thereof, such that the advantageous properties of the composition according to the invention are not, or are not substantially, adversely affected by the envisaged addition.

The said aqueous composition may form all or part of a cosmetic or dermatological composition which may thus moreover comprise a cosmetically or dermatologically acceptable medium, that is to say a medium that is compatible with use on keratin materials such as the skin, the nails, the hair, the eyelashes, the eyebrows, mucous membranes and semi-mucous membranes, and any other area of body or facial skin.

The said composition thus finds a particular application as a cosmetic make-up or care composition, which may be applied to keratin materials (the skin, the nails, the hair, the eyelashes, the eyebrows, mucous membranes and semi-mucous membranes, and any other area of body or facial skin).

A subject of the invention is also the cosmetic use of the composition according to the invention for making up and/or caring for keratin materials.

The invention is illustrated in greater detail in the examples which follow.

### EXAMPLE 1

The polymers with poly-N-vinylcaprolactam grafts may be obtained according to the following two-step process:

Synthesis of N-Vinylcaprolactam Oligomers Bearing an Amino Reactive End

26.4 g of N-vinylcaprolactam (0.189 mol) and 52 ml of methanol dried over molecular sieves are introduced into 250 ml three-necked round-bottomed flask sheltered from the light by means of an aluminium envelope and equipped with a condenser and a nitrogen inlet. This mixture is placed under a nitrogen sparge and is heated with stirring to 30° C. using a water bath. After 45 minutes, 1.73 g of aminoethanethiol hydrochloride (0.015 mol) predissolved in 20 ml of dried methanol are added to the reaction medium. 15 minutes later, 0.312 g of azobis(isobutyronitrile) (1.9×10⁻³ mol) dissolved in 20 ml of dried methanol is added to the reaction medium. This reaction medium is kept stirring under a nitrogen atmosphere for 3 hours at 60° C. The heating is then stopped and the reaction medium is kept stirring under a nitrogen sparge until it has cooled to room temperature.

The N-vinylcaprolactam oligomers synthesized are isolated by evaporating off the methanol and redissolving the product in water, followed by freeze-drying.

Grafting of the N-Vinylcaprolactam Oligomers onto Polyacrylic Acid

3 g of polyacrylic acid with a molar mass of 450 000 g/mol are dissolved in 125 ml of 1-methyl-2-pyrrolidone in a 500 ml three-necked round-bottomed flask equipped with a condenser, with stirring at 60° C. for 12 hours. 2.5 g of N-vinylcaprolactam oligomers predissolved in 125 ml of 1-methyl-2-pyrrolidone are introduced dropwise into the reaction medium with stirring. 15 minutes later, 0.687 g of dicyclohexylcarbodiimide predissolved in 50 ml of 1-methyl-2-pyrrolidone are introduced dropwise into the reaction medium with vigorous stirring. The reaction medium is kept stirring for 12 hours at 60° C.

The reaction medium is then cooled to 20° C., after which it is placed in a refrigerator at 4° C. for 24 hours. The dicyclohexylurea crystals formed are then removed by filtration. The polymer is then neutralized using 19 g of 35% sodium hydroxide (4-fold excess relative to the number of moles of acrylic acid), leading to its precipitation. After leaving to stand for 12 hours, the reaction medium is filtered so as to recover the precipitated polymer. This polymer is dried under vacuum at 35° C. for 24 hours.

9 g of solid are recovered, and are dissolved in 2 l of deionized water. This solution is ultrafiltered using a Millipore ultrafiltration system containing a membrane with a cutoff threshold set at 10 000 daltons. The solution thus purified is freeze-dried so as to collect the polymer in solid form.

4.5 g of polyacrylic acid (450 000 g/mol) grafted with poly-N-vinylcaprolactam are obtained.

The proportion by mass of units with an LCST in the final polymer is 38%.

The polymer thus obtained has a solubility in water, at 20° C., of at least 10 g/l.

### EXAMPLE 2

The polymers according to the invention are compared with polymers of the prior art described in WO-95/24430.

The absorbance of aqueous solutions comprising these polymers is measured, by UV-visible spectroscopy, at a wavelength equal to 500 nm, and at a temperature of 35° C. and of 40° C.

The transmittance is deduced therefrom according to the relationship: absorbance=−log transmittance.

The following results are obtained:

| | Transmittance | |
|---|---|---|
| | at 35° C. | at 40° C. |
| Comparison 1 at 0.2% by | 76% | 63% |
| weight in water | | |
| Comparison 2 at 0.2% by | 50% | 42% |
| weight in water | | |
| Polymer of Example 1 at 5% | 90% | 87% |
| by weight in water | | |

Comparison 1: block copolymer poly(N-isopropyl acrylamide) and polyacrylic acid 24/76 (FIG. 1 of WO-95/24430).
Comparison 2: block copolymer poly(N-isopropyl acrylamide) and polyacrylic acid 43/57 (FIG. 1 of WO-95/24430).

It is thus found that the polymer according to the invention gives compositions that are markedly more transparent than those of the prior art.

### EXAMPLE 3

A heat-induced gelling aqueous gel is prepared, comprising:

polymer of Example 1 (solids) 5 g;

water qs 100 g.

This composition is prepared by simple introduction of the polymer into saline water with stirring for 2 hours at 20° C.

The viscosity of the composition is measured, at 20° C. and at 40° C., using a Haake RS150 rheometer equipped with a cone/plate geometry 6 cm/2° and a temperature control system. The viscosity measurements are carried out in the flow mode by imposing a shear rate equal to 10 s⁻¹.

The following results are obtained:

viscosity at 20° C.: 0.010 Pa.s;

viscosity at 40° C.: 1 Pa.s.

### REFERENCES

[1] D. Hourdet et al., Polymer, 1994, Vol. 35, No. 12, pages 26-24-26-30.

[2] F. L'Alloret et al., Coll. Polym. Sci., 1995, Vol. 273, No. 12, pages 1163-1173.

[3] F. L'Alloret, Revue de l'Institut Francais du Pétrole [Review of the French Petroleum Institute], 1997, Vol. 52, No. 2, pages 117-128.

[4] EP-A-0 583 814.

[5] EP-A-0 629 649.

[6] WO-A-95 24430.

[7] WO-A-97 00275.

1. Polymer comprising water-soluble units and units having a temperature of LCST type, said polymer being obtainable by reaction between the reactive sites, firstly of the water-soluble units bearing, before reaction, at least two reactive sites, and secondly of the units with an LCST bearing, before reaction, at least one reactive site, so as to form a covalent bond, said units with an LCST consisting of N-vinylcaprolactam homopolymers or of copolymers derived therefrom, the proportion by mass of the units with an LCST in the polymer being between 5% and 70%.

2. Polymer according to claim 1, which is in the form of a block polymer comprising water-soluble blocks alternating with blocks with an LCST, or in the form of a grafted polymer whose backbone is formed from water-soluble units and bears grafts with an LCST, this structure possibly being partially crosslinked.

3. Polymer according to either of the preceding claims, in which the water-soluble units, before reaction, are obtained by free-radical polymerization of at least one monomer A chosen, alone or as a mixture, from:
(meth)acrylic acid;
vinyl monomers of formula (Ia) below: 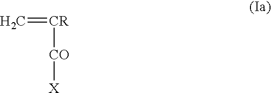
in which:
R is chosen from H, —CH₃, —C₂H₅ or —C₃H₇;
X is chosen from:
alkyl oxides of —OR′ type in which R′ is a linear or branched, saturated or unsaturated hydrocarbon-based radical containing from 1 to 6 carbons, substituted with at least one hydroxyl (—OH); primary amine (—NH₂); secondary amine (—NHR₁) or tertiary amine (—NR₁R₂) group, with R₁ and R₂, independently of each other, representing a linear or branched, saturated or unsaturated hydrocarbon-based radical containing 1 to 25 carbon atoms, with the proviso that the sum of the carbon atoms of R₁+R₂ does not exceed 26; a halogen atom (iodine, bromine, chlorine or fluorine);
groups —NH₂, —NHR′ and —NR′R″ in which R′ and R″ are, independently of each other, linear or branched, saturated or unsaturated hydrocarbon-based radicals containing 1 to 25 carbon atoms, with the proviso that the total number of carbon atoms of R′+R″ does not exceed 26, the said R′ and R″ optionally being substituted with a hydroxyl (—OH); sulphonic (—SO₃); sulphate (—SO₄); phosphate (—PO₄H₂); primary amine (—NH₂); secondary amine (—NHR₁), tertiary amine (—NR₁R₂) and/or quaternary amine (—N⁺R₁R₂R₃) group, with R₁, R₂ and R₃ being, independently of each other, a linear or branched, saturated or unsaturated hydrocarbon-based radical containing 1 to 25 carbon atoms, with the proviso that the sum of the carbon atoms of R₁+R₂ does not exceed 26, and that the sum of the carbon atoms of R₁+R₂+R₃ does not exceed 27;
maleic anhydride;
itaconic acid;
vinyl alcohol of formula CH₂═CHOH;
vinyl acetate of formula CH₂═CH—OCOCH₃.

4. Polymer according to claim 3, in which the water-soluble units, before reaction, are obtained by polymerization also of at least one monomer B chosen, alone or as a mixture, from
vinyl monomers of formula (Ib) below: 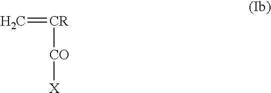
in which:
R is chosen from H, —CH₃, —C₂H₅ or —C₃H₇;
X is chosen from alkyl oxides of —OR′ type in which R′ is a linear or branched, saturated or unsaturated hydrocarbon-based radical containing from 1 to 6 carbons, optionally substituted with a sulphonic (—SO₃⁻), sulphate (—SO₄⁻), phosphate (—PO₄H₂); and/or quaternary amine (—N⁺R₁R₂R₃) group, with R₁, R₂ and R₃ being, independently of each other, a linear or branched, saturated or unsaturated hydrocarbon-based radical containing 1 to 25 carbon atoms, with the proviso that the sum of the carbon atoms of R₁+R₂+R₃ does not exceed 27;
N-vinyllactams such as N-vinylpyrrolidone, N-vinylcaprolactam and N-butyrolactam;
vinyl ethers of formula CH₂═CHOR in which R is a linear or branched, saturated or unsaturated hydrocarbon-based radical containing from 1 to 25 carbons;
styrene and its derivatives, especially styrene sulphonate;
dimethyldiallylammonium chloride;
vinylacetamide.

5. Polymer according either of claims 1 and 2, in which the water-soluble units, before reaction, are chosen from:
water-soluble polyurethanes having, before reaction, at least two reactive sites, especially bearing carboxylic acid functions;
xanthan gum;
alginates and derivatives thereof such as propylene glycol alginate;
cellulose derivatives and especially carboxymethylcellulose, hydroxypropylcellulose, hydroxyethylcellulose and quaternized hydroxyethylcellulose;
galactomanans and derivatives thereof, such as Konjac gum, guar gum, hydroxypropylguar, hydroxypropylguar modified with sodium methylcarboxylate groups, and hydroxypropyl trimethylammonium guar chloride;
polyethyleneimine.

6. Polymer according to one of the preceding claims, in which the water-soluble units have a molar mass of between 5 000 g/mol and 5 000 000 g/mol when they constitute the water-soluble backbone of a grafted polymer; or a molar mass of between 5 000 g/mol and 100 000 g/mol when they constitute a block of a multiblock polymer.

7. Polymer according to one of the preceding claims, in which the units with an LCST consist of N-vinylcaprolactam homopolymers of formula (II) below: 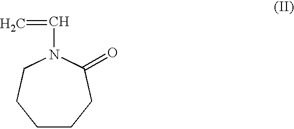
or of copolymers of N-vinylcaprolactam and:
of a vinyl monomer of formula (III) below: 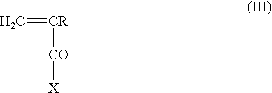
in which:
R is chosen from H, —CH₃, —C₂H₅ or —C₃H₇; and
X is chosen from:
alkyl oxides of —OR′ type in which R′ is a linear or branched, saturated or unsaturated hydrocarbon-based radical containing from 1 to 6 carbons, optionally substituted with at least one halogen atom (iodine, bromine, chlorine or fluorine); a sulphonic (—SO₃⁻), sulphate (—SO₄⁻), phosphate (—PO₄H₂); hydroxyl (—OH); primary amine (—NH₂); secondary amine (—NHR₁), tertiary amine (—NR₁R₂) or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R₁+R₂+R₃ does not exceed 7; and
groups —NH₂, —NHR′ and —NR′R″ in which R′ and R″ are, independently of each other, linear or branched, saturated or unsaturated hydrocarbon-based radicals containing 1 to 6 carbon atoms, with the proviso that the total number of carbon atoms of R′+R″ does not exceed 7, the said R′ and R″ optionally being substituted with a halogen atom (iodine, bromine, chlorine or fluorine); a hydroxyl (—OH); sulphonic (—SO₃⁻), sulphate (—SO₄⁻); phosphate (—PO₄H₂); primary amine (—NH₂); secondary amine (—NHR₁), tertiary amine (—NR₁R₂) and/or quaternary amine (—N⁺R₁R₂R₃) group with R₁, R₂ and R₃ being, independently of each other, a linear or branched, saturated or unsaturated hydrocarbon-based radical containing 1 to 6 carbon atoms, with the proviso that the sum of the carbon atoms of R′+R″+R₁+R₂+R₃ does not exceed 7;
maleic anhydride;
itaconic acid;
vinyl alcohol of formula CH₂═CHOH; vinyl acetate of formula CH₂═CH—OCOCH₃;
a vinyl ether of formula CH₂═CHOR in which R is a linear or branched, saturated or unsaturated hydrocarbon-based radical containing from 1 to 6 carbons;
styrene or derivatives thereof, especially styrene sulphonate;
dimethyldiallylammonium chloride; and
vinylacetamide.

8. Polymer according to one of the preceding claims, in which the molar mass of the units with an LCST is between 1 000 and 500 000 g/mol and especially between 2 000 and 50 000 g/mol.

9. Polymer according to one of the preceding claims, in which the units with an LCST, before reaction, are in the form of N-vinylcaprolactam homopolymers or of amino, especially monoamino, diamino or triamino, derivative copolymers.

10. Polymer according to one of the preceding claims, in which the proportion by mass of the units with an LCST in the final polymer is preferably between 20% and 65%, and particularly between 30% and 60% by weight relative to the final polymer.

11. Polymer according to one of the preceding claims, in which the heat-induced demixing temperature of LCST type of the said units with an LCST is between 5° C. and 40° C. and preferably 10° C. and 35° C., for a concentration by mass in water of 1% by weight of the said units with an LCST.

12. Polymer according to one of the preceding claims, having a solubility in water, at 20° C., of at least 10 g/l and preferably of at least 20 g/l.

13. Polymer according to one of the preceding claims, for producing a thickened, or even gelled, transparent aqueous composition, which has a maximum light transmittance value, irrespective of the wavelength of between 400 and 800 nm, through a sample 1 cm thick, of at least 80% and preferably of at least 85%.

14. Aqueous composition comprising at least one polymer as defined according to one of the preceding claims, and an aqueous phase.

15. Composition according to claim 14, in which the polymers are present in an amount of between 0.01% and 20% by weight, especially from 0.05% to 15% by weight and in particular from 0.1% to 10% by weight.

16. Composition according to either of claims 14 and 15, moreover comprising a cosmetically or dermatologically acceptable medium.

17. Cosmetic use of the composition according to any one of claims 14 to 16, for making up and/or caring for keratin materials.
